(19) United States
(12) Patent Application Publication
(10) Pub. No.: US 2026/0105986 A1
(43) Pub. Date: Apr. 16, 2026

(54) Nucleotide sequencing data compression

(71) Applicant: Keygene N.V., Wageningen (NL)

(72) Inventors: Cameron Sean Johnson, Rockville, MD (US); Stephen Ezra Schauer, Rockville, MD (US)

(73) Assignee: Keygene N.V., Wageningen (NL)

(21) Appl. No.: 19/139,885

(22) PCT Filed: Dec. 22, 2023
(86) PCT No.: PCT/EP2023/087628

§ 371 Date: Jun. 17, 2025

Related U.S. Application Data
(60) Provisional application No. 63/476,645, filed on Dec. 22, 2022.

(30) Foreign Application Priority Data
Dec. 22, 2022 (EP) 22215833.9

(51) Int. Cl. G16B 30/00; G16B 40/20; G16B 45/00

(52) U.S. Cl. CPC G16B 30/00; G16B 40/20; G16B 45/00

(57) ABSTRACT

The present invention relates to the field of genetics, more in particular to quantitative sequencing data. A method of processing and/or compressing quantitative sequencing data is provided, a computer-readable storage medium to comprise such processed data and a computing device comprising at least one processor configured to process such data.

### FIELD OF THE INVENTION

The invention is in the field of analysing and processing biological datasets, preferably large biological datasets. Such large datasets are preferably nucleotide datasets obtained by high-throughput sequencing. These nucleotide datasets can generated by high-throughput sequencing of genome(s) or transcriptome(s) of one or more samples.

### BACKGROUND

The genotype of an organism is determined by genetic material inherited from parents to offspring comprising the genome that codes for all processes in the organism's life. In eukaryotic organisms this genetic material encompasses double-stranded DNA strings constituting chromosomes that are located in the nuclei of the organism's cells. The phenotype is understood as the organism's observable characteristics. In plant and animal biology, these observable characteristics are indicated as traits. The organism's phenotypes are largely determined by the RNA and/or proteins encoded by the genome and their expression levels. These RNAs and/or proteins are encoded on loci in the genome indicated as genes.

Genes are regions of DNA that can be considered as units of transcription. Although some genes are transcribed into RNA not encoding for proteins (indicated as noncoding RNA), genes in general refer to structures comprising a coding sequence composed of one or more exons that may together be translated into a protein. The exons are preceded and followed by regulatory sequences and optionally (in eukaryotic cells) interrupted by introns. Gene expression comprises the process of transcribing the gene to form a pre-messenger RNA (pre-mRNA) that comprises the string of exons interrupted by introns and short pieces of the regulatory sequences indicated as 5′-UTR and 3′-UTR, respectively. By a process called post-transcriptional modification, the possible introns are spliced out and a 5-cap and polyadenylated-tail (in short: poly A-tail) are added to the respective 5′ and 3′ end of the mRNA thereby forming mature mRNA. This mature mRNA may subsequently be translated into a protein.

In order to understand and possibly even predict an organism's phenotype from its genotype, it is relevant to have an understanding of the sequences coding for RNAs and/or proteins and the (non-expressed) sequences that regulate gene expression. Next-generation sequencing techniques have allowed to unravel the sequence of the genome of many species. Because of the uniqueness of genomic sequences, next generation sequencing techniques have proven their value in many more applications, such as e.g., forensics, hereditary or infectious disease diagnostics, and analysis of biodiversity. Comparative genomics between related species has been used to unravel genetic sequences based on a high level of sequence conservation between related species. Quantitative sequencing methods have provided an extra dimension. For instance, understanding the amount of genomic DNA of a species, and/or the abundance of specific transcripts of an individual, present in a sample is of interest in various areas such as forensics, medical diagnostics and agricultural sciences. Most of these techniques rely on read depths of next-generation sequencing (e.g. see Reinecke et al. BMC Bioinformatics. 2015; 16(17)), wherein the read depth of a particular nucleotide sequence reflects the abundance of the originating nucleic acid being present in the sample, which in turn may reflect e.g., the abundance of a particular microbe present in an environmental sample, chromosomal copy number present in a sample for prenatal screening, or the number of tumour cells present a biopsy sample. Apart from DNA sequencing, quantitative sequencing methods relying on read depths are also available for quantification of RNA transcripts, indicated as ‘RNA-seq’. RNA-seq is the deep-sequencing of cellular RNA. It is a quantitative method that can be used to determine mature mRNA expression levels, and therewith expression of genes within a cell, without the requirement of prior sequence knowledge of these genes (Want et al. Nat Rev Genet. 2009; 10(1): 57-63). In addition to polyadenylated messenger RNA (mRNA) transcripts, RNA-Seq can be applied to investigate different populations of RNA, including total RNA, pre-mRNA, and noncoding RNA (ncRNA), such as ribosomal RNA (rRNA) and transfer RNA (TRNA) both involved in RNA translation, small nuclear RNA (snRNA) involved in splicing, small nucleolar RNAs (snoRNA) involved in the modification of ribosomal RNA, micro RNA (miRNA), trans-acting siRNA (tasiRNA) and piwi-interacting RNA (piRNA) both regulating gene expression at the posttranscriptional level and long noncoding RNA (InoRNA) involved in chromatin remodelling, transcriptional control, and posttranscriptional processing (Kukurba and Montgomery, Cold Spring Harb Protoc 2015; 11:951-969). In RNA-seq, RNA is extracted and, depending on the protocol used, subsets of RNA molecules are isolated, such a mature mRNAs by enrichment for polyadenylated transcripts. The isolated RNA molecules are reverse transcribed to cDNA fragments, optionally amplified, and subsequently sequenced. These sequencing reads may then be aligned back to a pre-sequenced reference genome or reference transcripts, or in some cases assembled without the reference (Oshlack et al. Genome Biol 2010; 11(12): 220; Wang et al. Nat Rev Genet 2009; 10(1): 57-63. and Auer et al. Brief Funct Genomics 2012; 11(1): 57-62).

RNA-seq data can be used for studying the transcriptome also indicated as transcriptomics. Transcriptome analysis may provide detailed and quantitative information on, amongst others, gene expression, alternative splicing and allele-specific expression. Mature mRNA levels, e.g. expressed per cell, have been the most frequently studied RNA species because they encode proteins. (Ciaran Evans, Johanna Hardin and Daniel M. Stoebel Selecting between-sample RNA-Seq normalization methods from the perspective of their assumptions Briefings in Bioinformatics, 19(5), 2018, 776-79).

Recent advances in the sequencing workflows, from sample preparation and sequencing platforms to bioinformatic data analysis, enabled the use of quantitative sequencing methods in an advanced way in many different disciplines ranging from evolutionary research to medical applications. However, despite these tools available, these datasets are costly and their massive nature makes analysis processes slow and requiring large data storage capacity. Therefore, there is a need for improved ways to use available and/or obtained quantitative sequencing datasets.

### SUMMARY

The invention may be summarized in the following embodiments:

Embodiment 1. A method of processing a nucleotide-sequencing dataset, wherein the method comprises the steps of:
(a) providing the nucleotide-sequencing dataset comprising read depth values assigned to nucleotide sequences;
(b) providing a reference nucleotide sequence;
(c) converting the read depth values present in the nucleotide-sequencing dataset to single characters indicating Relative Read Depths (RRDs) by ordinal class assignment; and
(d) mapping the single characters to the reference nucleotide sequence of (b) at base-pair resolution, wherein the method is preferably a computer-implemented method.

Embodiment 2. The method of embodiment 1, wherein the nucleotide-sequencing dataset of (a) is a transcriptomic dataset obtained by RNA-seq.

Embodiment 3. The method of embodiment 1, wherein the nucleotide-sequencing dataset of (a) is a genomic dataset obtained by DNA sequencing.

Embodiment 4. The method of any one of the preceding embodiments, wherein the reference nucleotide sequence is a genomic sequence, preferably a whole genomic sequence.

Embodiment 5. The method of any one of the preceding embodiments, wherein step (a) comprises the following (sub-)steps:
(1) providing a nucleic acid sample;
(2) optionally enriching and/or isolating a subset of nucleic acid molecules from at least part of said nucleic acid sample;
(3) preparing a sequencing library of the nucleic acid molecules of (1) or the enriched and/or isolated nucleic acid molecules of (2);
(4) obtaining nucleotide sequences by sequencing the sequencing library of (3); and
(5) assigning read depth values to the nucleotide sequences obtained in (4), thereby obtaining a nucleotide-sequencing dataset comprising read depth values assigned to the nucleotide sequences of the sequenced nucleic acid molecules.

Embodiment 6. The method of embodiment 5, wherein the nucleic acid molecules of (1) and optionally the enriched and/or isolated nucleic acid molecules of (2) are mRNA molecules, and wherein the method further comprises a (sub-)step of converting the mRNA to cDNA prior to step (3) of preparing a sequencing library of said cDNA.

Embodiment 7. The method of any one of the preceding embodiments, wherein the reference nucleotide sequence of (b) is obtained by (whole)genomic sequencing.

Embodiment 8. The method of any one of the preceding embodiments, wherein in step (c) the read depth values are classified in N+1 consecutive RRDs, wherein each RRD comprises a read depth upper boundary, wherein a read depth value is assigned to the lowest ranked RRD for which said read depth value does not exceed the read depth upper boundary of said RRD, wherein the read depth upper boundary of the lowest ranked RRD is 0, and wherein for the remaining RRDs the read depth upper boundary is given by:

$$UB = f \cdot {x}^{RV} \qquad ( Formula 2 )$$

wherein,
UB is the read depth upper boundary of the given RRD;
x is a base that preferably has a value between 1 and Euler's number (e);
f is a scaling factor that preferably has a value of at least 1;
RV is the given ranking value selected from the group consisting of {1, 2, 3, 4, . . . N} of the RRD for which the upper boundary is calculated; and
N is the ranking value of the highest ranked RRD.

Embodiment 9. The method according to embodiment 8, wherein the value of the base x is:

$$x = {( MRD / f )}^{1 / N} \qquad ( Formula 4 )$$

wherein:
x is the base of Formula 2 and 3;
MRD is the maximum read depth; and
N is the ranking value of the highest ranked RRD.

Embodiment 10. The method according to embodiment 8 or 9, wherein the value of the scaling factor f is given by:

$$f = MRD / {e}^{N} \qquad ( Formula 5 )$$

wherein:
f is a scaling factor of Formula 2 and 4;
MRD is the maximum read depth;
e is Euler's number; and
N is the ranking value of the highest ranked RRD.

Embodiment 11. The method according to any one of embodiments 8-10, wherein f is provided by Formula 5 in case MRD≥e^{N}, and f=1 in case MRD<e^{N}, and wherein the read depth upper boundary of the lowest ranked RRD is 0, and wherein for the remaining RRDs the read depth upper boundary is given by:

$$UB = ( MRD \cdot {e}^{RV} ) / {e}^{N} \qquad ( Formula 6 )$$

in case MRD≥e^{N}; and by

$$UB = {( MRD )}^{RV / N} \qquad ( Formula 8 )$$

in case MRD<e^{N},
wherein
UB is the read depth upper boundary of the given RRD;
MRD is the maximum read depth;
e is Euler's number;
RV is the given ranking value selected from the group consisting of {1, 2, 3, 4, . . . N} of the RRD for which the upper boundary is calculated; and N is the ranking value of the highest ranked RRD.

Embodiment 11. The method according to any one of the preceding embodiments, wherein said method is performed on multiple samples.

Embodiment 12. Use of processed nucleotide-sequencing data obtained by or obtainable by any one of the methods of embodiments 1-11, in a computer implemented method, preferably for predicting genomic sequences and/or for comparing gene expression patterns within or between samples.

Embodiment 13. Use of embodiment 12, wherein said computer implemented method comprises machine learning.

Embodiment 14. A computer-readable storage medium comprising the processed nucleotide-sequencing data obtained by the method of any one of embodiments 1-11.

Embodiment 15. A computing device comprising at least one processor, wherein the processor is configured to perform any of the method in embodiments 1 to 11.

### LEGEND TO THE FIGURES

FIG. 1 illustrates a method of processing a nucleotide-sequencing dataset.

FIG. 2 illustrates a method of obtaining a nucleotide-sequencing dataset comprising read depth values assigned to nucleotide sequences.

FIG. 3 illustrates a method for predicting genes and gene expression levels of a nucleic acid sample.

FIG. 4 illustrates a computing device for performing the present invention.

### DEFINITIONS

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al. Molecular Cloning. A Laboratory Manual, 2^{nd} Edition, Cold Spring Harbor Laboratory Press, Cold Spring Harbor, N. Y., 1989; Ausubel et al. Current Protocols in Molecular Biology, John Wiley & Sons, New York, 1987 and periodic updates; and the series Methods in Enzymology, Academic Press, San Diego.

“A,” “an,” and “the”: these singular form terms include plural referents unless the content clearly dictates otherwise. Thus, for example, reference to “a cell” includes a combination of two or more cells, and the like.

As used herein, the term “about” is used to describe and account for small variations. For example, the term can refer to less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

“Amplification” used in reference to a nucleic acid or nucleic acid reactions, refers to in vitro methods of making copies of a particular nucleic acid, such as a target nucleic acid, and/or a tagged nucleic acid. Numerous methods of amplifying nucleic acids are known in the art, and amplification reactions include, but is not limited to, polymerase chain reactions, ligase chain reactions, strand displacement amplification reactions, rolling circle amplification reactions, transcription-mediated amplification methods such as NASBA (e.g., U.S. Pat. No. 5,409,818), loop mediated amplification methods (e.g., “LAMP” amplification using loop-forming sequences, e.g., as described in U.S. Pat. No. 6,410,278) and isothermal amplification reactions. The nucleic acid that is amplified can consist of, or derived from DNA or RNA or a mixture of DNA and RNA, including modified DNA and/or RNA. The products resulting from amplification of a nucleic acid molecule or molecules (i.e., “amplification products”), whether the starting nucleic acid is DNA, RNA or both, can be either DNA or RNA, or a mixture of both DNA and RNA nucleosides or nucleotides, or they can comprise modified DNA or RNA nucleosides or nucleotides.

The term “nucleic acid sample” as used herein and also indicated herein as “sample” describes a sample comprising one or more nucleic acids, preferably genomic DNA molecules and/or RNA molecules expressed from a genome, wherein the expressed RNA molecules are also indicated herein as “the transcriptome”. A nucleic acid sample may be a biological sample originating from one or more biological sources, and may be obtained from one or more of the same or different individuals, e.g. of human, plant, animal, bacteria, fungus, algae, insect, etc. The biological sample may be from a cell, tissue, biopsy or bodily fluid. Optionally, the nucleic acid sample is an environmental sample, such as a sample from soil or water, comprising mixtures of nucleic acids, optionally originating from different species in the form of e.g., a whole organism (for example bacteria or viruses) and/or an organisms' blood, urine, faeces, gametes and/or skin. The sample may contain a mixture of material, typically, although not necessarily, in liquid form, containing one or more RNA molecules or RNA-transcripts.

“Comprising” is construed as being inclusive and open ended, and not exclusive. Specifically, the term and variations thereof mean the specified features, steps or components are included. These terms are not to be interpreted to exclude the presence of other features, steps or components.

The terms “double-stranded” and “duplex” as used herein, describes two complementary polynucleotides that are base-paired, i.e., hybridized together. Complementary nucleotide strands are also known in the art as reverse-complement.

“Expression”: this refers to the process wherein a DNA region, which is operably linked to appropriate regulatory regions, particularly a promoter, is transcribed into an RNA. In case of a protein-encoding RNA, the RNA may in turn be translated into a protein or peptide.

The term “gene” means a DNA fragment comprising a region (transcribed region), which is transcribed into an RNA molecule (e.g. a pre-mRNA or noncoding RNA) in a cell by an RNA polymerase enzyme in a process called transcription. The transcribed region that may be translated in a protein may be called an open reading frame (ORF) and starts with a three letter code indicated as the start codon, and ends with one of the three possible stop codons. The ORF may comprise exons and one or more introns. Upstream of the ORF is a 5′UTR and downstream a 3′UTR that make up the boundaries of the transcribed RNA. For pre-mRNA, during maturation, the introns may be spliced out and a 5-cap and polyadenylated-tail (in short: polyA tail) are converted to the respective 5′ and 3′ end of the RNA thereby forming mature mRNA. Hence mature mRNA consists of the following nucleotide sequence elements: a 5′UTR, exons, a 3′UTR and a polyA tail. The exons together make up the coding sequence (CDS). The mature mRNA may subsequently be translated in a protein in a process called translation. The ORF is associated with (or operably linked to) untranscribed and/or untranslated regulatory sequences at its 5′- and/or 3′-end such as the promoter sequence that can bind transcription factors that recruit and help the RNA polymerase to start transcription. Apart from the promoter sequence, regulatory sequences may act as enhancer and/or silencers of transcription for instance by binding certain enhancer or inhibiting elements and/or by influencing the chromatin structure.

The term “nucleotide” includes, but is not limited to, naturally occurring nucleotides, including guanine, cytosine, adenine, thymine and uracil (G, C, A, T and U, respectively). The term “nucleotide” is further intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “nucleic acid”, “polynucleotide” and “nucleic acid molecule” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein). The nucleic acid may hybridize with naturally occurring nucleic acids in a sequence specific manner analogous to that of two naturally occurring nucleic acids, e.g., can participate in Watson-Crick base pairing interactions. In addition, nucleic acids and polynucleotides may be isolated (and optionally subsequently fragmented) from cells, tissues and/or bodily fluids. The nucleic acid can be e.g. genomic DNA (gDNA), mitochondrial, cell free DNA (cfDNA), DNA from a sequencing library and/or RNA from a sequencing library. The nucleic acid is preferably a double-stranded molecule, unless it is clear from its context that a single-stranded molecule is intended.

The term “oligonucleotide” as used herein denotes a single-stranded multimer of nucleotides, preferably of about 2 to 200 nucleotides, or up to 500 nucleotides in length. Oligonucleotides may be synthetic or may be made enzymatically, and, in some embodiments, are about 10 to 50 nucleotides in length. Oligonucleotides may contain ribonucleotide monomers (i.e., may be oligoribonucleotides) or deoxyribonucleotide monomers. An oligonucleotide may be about 10 to 20, 20 to 30, 30 to 40, 40 to 50, 50 to 60, 60 to 70, 70 to 80, 80 to 100, 100 to 150, 150 to 200, or about 200 to 250 nucleotides in length, for example.

“Sequence” or “Nucleotide sequence”: This refers to the order of nucleotides of, or within a nucleic acid. In other words, any order of nucleotides in a nucleic acid may be referred to as a sequence or nucleic acid sequence. For example, the target sequence is an order of nucleotides comprised in a single strand of a DNA duplex.

“Sequence identity” is herein defined as a relationship between two or more amino acid (polypeptide or protein) sequences or two or more nucleotide (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods. The percentage sequence identity/similarity can be determined over the full length of the sequence.

“Sequence identity” and “sequence similarity” can be determined by alignment of two amino acid or two nucleotide sequences using global or local alignment algorithms, depending on the length of the two sequences. Sequences of similar lengths are preferably aligned using a global alignment algorithm (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall lengths, local alignments, such as those using the Smith Waterman algorithm, are preferred.

Alternatively percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, word length=12 to obtain nucleotide sequences homologous to nucleic acid molecules of the invention. BLAST protein searches can be performed with the BLASTx program, score=50, word length=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

The term “sequencing,” as used herein, refers to a method by which the identity of at least about 10 consecutive nucleotides (e.g., the identity of at least about 20, at least about 50, at least about 100 or at least about 200 or more consecutive nucleotides) of a polynucleotide are obtained. The term “next-generation sequencing” refers to the so-called parallelized sequencing-by-synthesis or sequencing-by-ligation platforms, e.g., such as currently employed by Illumina, Life Technologies, PacBio, Roche and Complete Genomics etc. Next-generation sequencing methods may also include nanopore sequencing methods, such as those commercialized by Oxford Nanopore Technologies, or electronic-detection based methods such as Ion Torrent technology commercialized by Life Technologies.

### DETAILED DESCRIPTION

In a first aspect, provided is a method of processing nucleotide-sequencing data, wherein the method comprises the steps of:
(a) providing a nucleotide-sequencing dataset comprising read depth values assigned to nucleotide sequences;
(b) providing a reference nucleotide sequence;
(c) converting the read depth values present in the nucleotide-sequencing dataset to single characters indicating Relative Read Depths (RRDs) by ordinal class assignment; and
(d) mapping the single characters to the reference nucleotide sequence of (b) at base-pair resolution.

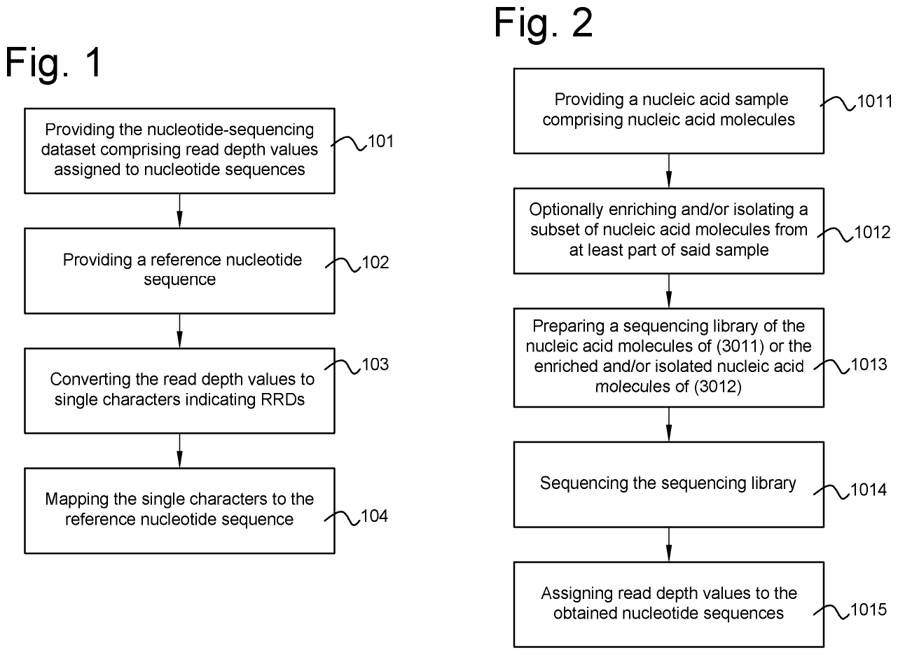

FIG. 1 depicts a flowchart illustrating said method, having a step 101 of providing a nucleotide-sequencing dataset comprising read depth values assigned to nucleotide sequences, a step 102 of providing a reference nucleotide sequence, a step 103 of converting the read depth values present in the nucleotide-sequencing dataset to single characters indicating Relative Read Depths (RRDs) by ordinal class assignment, and a step 104 of mapping these single characters to the reference nucleotide sequence of (b) at base-pair resolution. Hence in FIG. 1, step 101 corresponds to step (a) as described herein, step 102 corresponds to step (b) as described herein, step 103 corresponds to step (c) as described herein and step 104 corresponds to step (d) as described herein.

The nucleotide-sequencing dataset of (a) is preferably the output of a nucleotide sequencing method, preferably the output of sequencing nucleic acid molecules of a sample. Preferably, the nucleotide-sequencing dataset of (a) is preferably obtained by quantitative sequencing. Alternatively, the dataset is from a public database. As specified in (a), the sequencing dataset comprises read depth values assigned to nucleotide sequences. Read depth value, also indicated herein as read depth, is to be understood herein as the number of times a given nucleotide or nucleotide sequence has been read in a nucleotide-sequencing method, for instance by sequencing nucleic acid molecules of a sample. The dataset of step (a) comprises read depth values assigned to a nucleotide sequence, preferably of nucleic acid molecules present in a sample. As each read depth value may be indicative for the quantity of each of the corresponding nucleic acid molecule present in the sample, the nucleotide-sequencing dataset comprising read depth values may also be indicated as a quantitative nucleotide-sequencing dataset.

Provided is a method that optimally converts the read depth values of one or more nucleotide-sequencing datasets to single characters that is of itself a class assignment but may also be used later as a ground truth for ordinal classification, preferably while retaining as much of the information of the nucleotide-sequencing dataset data as possible. “Ground truth” is to be understood herein as the reality to be modelled such as with machine learning. The conversion of read depth values into (ordinal) RRDs may also be indicated as binning, and the RRDs may be indicated as bins. Other names that may be given to a bin is an ordinal variable or a category. By ordinal class assignment, the read depth values are converted to ordinal variables or categories that can be ranked from low to high. In the method provided herein, these categories, bins or RRDs are indicated by single characters that each represent a consecutive range of read depth values. Hence, the read depth values are grouped in RRDs, and each read depth value within a certain range of read depth values is assigned to the same RRD. Each RRD has a certain read depth upper boundary (or upper limit), meaning that read depths exceeding said upper boundary will be assigned to a higher ranked RRD. Likewise, each RRD has a certain read depth lower boundary, meaning that read depths having a value that is equal to or lower than said read depth lower boundary (or lower limit) will be assigned to a lower ranked RRD.

In case the number of different RRDs and hence the number of single characters in step (c) are less than the number of different read depth values present in the dataset provided in (a), step (c) of the method provided herein results in data compression, and the method indicated above can then be phrased as a method of compressing nucleotide-sequencing data of a nucleic acid sample.

The nucleotide-sequencing dataset of step a) comprises read depth values assigned to nucleotide sequences. In step c) each of these read depth values is assigned to a certain RRD. By aligning these nucleotide sequences or their complements to the one or more reference nucleotide sequences of b), each of the nucleotides of said one or more reference nucleotide sequences can be assigned an RRD in step c); this process is indicated herein as mapping RRDs to a reference nucleotide sequence on base-pair resolution. “Base-pair resolution” is to be understood herein as assigning an RRD to each base pair of a double-stranded DNA sequence or assigning an RRD to each base or nucleotide of a single-stranded DNA or RNA, optionally being one strand of genomic DNA.

Optionally, read depth values of the nucleotide-sequencing dataset are converted to RRDs prior to mapping the single characters representing the RRDs per nucleotide to a reference nucleotide sequence. Alternatively, the read depth values of the nucleotide-sequencing dataset are mapped per nucleotide to a reference nucleotide sequence thereby rendering a string of read depth values, and subsequently the values of the string are converted to RRDs. In both cases, the result of step (d) is a nucleotide sequence represented by a single character per nucleotide indicating its RRD in said reference nucleotide sequence, which is also indicated herein as a string of RRDs mapped to a reference nucleotide sequence or RRDs mapped to a reference nucleotide sequence at base-pair resolution.

Optionally, the reference nucleotide sequence of b) is a genomic sequence and the nucleotide sequences of the dataset are transcripts or RNAs, preferably mRNAs, more preferably mature mRNA, and the nucleotide-sequencing dataset is an RNA-seq dataset or transcriptomic dataset. In case the dataset is an RNA-seq dataset, the read depth values may correspond to transcript expression level values. As each RNA-nucleotide of an RNA sequence is transcribed from a particular DNA-nucleotide of the genome, such DNA-nucleotide can be assigned the particular single character representing the RRD of the transcript encoded by said DNA-nucleotide. The RRDs, and hence the single characters indicating the RRDs, represent expression categories as deduced from the transcriptomic data, which can be assigned to the genome on base-pair resolution.

Preferably, the dataset of step (a) comprises nucleotide sequences, wherein the nucleotide sequences have been assigned read depth values. At least part of the sequences of step (a) may correspond to a reference nucleotide sequence of step (b). Hence preferably, at least part of the sequences of the dataset of step (a) have at least 50%, 60%, 70%, 80%, 90%, 95% or 100% identity with a reference sequence of step (b).

Preferably, the reference nucleotide sequence of (b) relates to the sequences of the dataset of (a). In particular, one or more, optionally all, of the nucleotide sequences of the dataset of (a) or their complements align to at least part of at least the reference nucleotide sequence of (b). In other words, one or more nucleotide sequences of the dataset of (a) or their complements show at least 50%, 60%, 70%, 80%, 90%, 95% or 100% identity with (a part of) at least one of said reference nucleotide sequence, over their whole length. Optionally, one or more of the nucleotide sequences of the dataset of (a) or their complements fully align to at least part of at least one of the reference nucleotide sequences of (b).

Optionally, at least one (optionally all) of the reference nucleotide sequences of (b) is a whole or part of a genomic nucleotide sequence. Said genomic sequence may be a cellular or an organellar genomic sequence, such as a nuclear, a mitochondrial or chloroplast genomic sequence.

A part of a genomic sequence may be a sequence of particular interest, for instance a sequence that is highly indicative for a particular species from which said genomic sequence is originating, or a particular sequence comprising a gene of interest, such as, but not limited to, a chromosomal region, a marker region, a conserved region, a (hyper) variable region, a Quantitative Trait Locus (QTL), or a specific gene relating to a particular crop trait, or a particular animal or human disease. Optionally, said reference genomic sequence of step (b) is of a particular species or individual. Optionally, in step (b) multiple reference sequences are provided, optionally of multiple species or individuals. Said reference sequence(s) may be from a public database or obtained by sequencing of a nucleic acid sample.

Optionally, the one or more reference nucleotide sequences of (b) and the nucleotide-sequencing dataset of (a) are obtained by sequencing the same nucleic acid sample, or by sequencing a nucleic acid sample of the same species. The nucleotide sequences of the dataset of (a) may be sequences of nucleic acid molecules that are fragments, amplicons and/or transcripts of the (genomic, ribosomal or mitochondrial) reference nucleotide sequences of (b). For instance, in case a reference nucleotide sequence is a nucleotide sequence of a genomic nucleic acid, the nucleotide-sequencing dataset in step (a) may be obtained from RNA transcripts from said genomic nucleic acid.

Converting read depth values of (quantitative) DNA- and/or RNA-sequencing datasets to single characters mapped to a reference sequence at base-pair resolution allows to further process and/or to analyse high volumes of such compressed data for instance in deep learning, machine learning and/or artificial intelligence processes and/or applications. This allows to tackle a highly diverse set of (quantitative) nucleic acid related questions ranging from evolutionary research questions, to medical applications, while requiring significantly reduced processing time and space.

Step (a) of the method provided herein may comprise the following (sub-)steps:
(1) providing a nucleic acid sample;
(2) optionally enriching and/or isolating a subset of nucleic acid molecules from at least part of said sample;
(3) preparing a sequencing library of said nucleic acid molecules of (1) or the enriched and/or isolated nucleic acid molecules of (2); and
(4) obtaining nucleotide sequences by sequencing the sequencing library of (3) and
(5) assigning read depth values to the nucleotide sequences obtained in (4), thereby producing a nucleotide-sequencing dataset comprising both nucleotide sequences and read depth values assigned to said nucleotide sequences.

FIG. 2 depicts a flowchart illustrating said method, having a step 1011 of providing a nucleic acid sample comprising nucleic acid molecules, optionally a step 1012 of enriching and/or isolating a subset of nucleic acid molecules from at least part of said sample, a step 1013 of preparing a sequencing library of the nucleic acid molecules of (1011), or the enriched and/or isolated nucleic acid molecules of (1012), a step 1014 of sequencing the sequencing library, and a step 1015 of assigning read depth values to the nucleotide sequences obtained in step 1014. Hence in FIG. 2, step 1011 corresponds to step (a)(1) as described herein, step 1012 corresponds to step (a)(2) as described herein, step 1013 corresponds to step (a)(3) as described herein, step 1014 corresponds to step (a)(4) as described herein and step 1015 corresponds to step (a)(5) as described herein.

The nucleic acid sample of the method provided herein may be from a single individual such as, but not limited to, a human, an animal, a plant, a fungus, an insect, a virus or a microbe. Said sample may be a cellular sample, a tissue sample and/or a liquid biopsy sample. The sample may be a sample of an individual of a population and/or a sample of a particular part of the individual, such as, but not limited to, a leaf, fruit or root sample of a plant, or of blood, saliva, cancerous tissue, urine or faeces of an animal or human and/or a sample collected from an individual under particular circumstances such as, but not limited to, a sample from a plant exposed to biotic stress, or a sample from a human or animal under medical treatment. Optionally, the nucleic acid sample is a pooled sample, optionally from different individuals or from the same individual. Optionally these multiple individuals are of different species. Optionally the nucleic acid sample is an environmental sample, such as, but not limited to a sample of soil or wastewater. In case the nucleic acid sample is a human or animal sample, the step of sample collection is preferably non-invasive. Preferably, the step of sample collection is not part of the method described herein. Preferably, the method provided herein is an ex vivo and/or in vitro method.

The nucleotide-sequencing dataset of (a) may be a DNA-sequencing dataset, preferably a quantitative DNA-sequencing dataset. Optionally, said dataset is obtained from a nucleic acid sample comprising two or more DNA molecules. In addition or alternatively, nucleotide-sequencing dataset of (a) may be an RNA-sequencing dataset, preferably a quantitative RNA-sequencing dataset. Optionally, said dataset is obtained from a nucleic acid sample as defined herein comprising two or more RNA molecules.

Optionally, the nucleotide-sequencing dataset of (a) comprises nucleotide sequences of two or more (different) individuals, species and/or cell types. Preferably, the read depth values of said dataset reflect the quantity of the nucleic acids within the nucleic acid sample, e.g. the quantity of nucleic acids of the two or more (different) individuals, species and/or cell types present in the sample. For instance, said sample may be an environmental sample or a sample of a plant, animal and/or human comprising nucleic acid molecules of multiple different microbes. Non-limiting examples of environmental samples are: soil, drink water, surface water and sewage water. Non-limiting examples of a sample of a plant, animal and/or human are: plant parts, blood, skin, saliva, gut (gastric juice), urine and faeces. Preferably in step (b), a reference sequence of these different individuals, species and/or cell types (optional of different microbes) are provided. Preferably, the reference nucleotide sequences are genomic sequences of the different individuals, species and/or cell types. Optionally, the reference sequences are RNA sequences of the different individuals, species and/or cell types. The nucleotide-sequencing dataset may be a (quantitative) DNA or RNA sequencing dataset. Within this embodiment, the single characters indicating RRDs are mapped to one of the multiple reference nucleotide sequences by aligning the nucleotide sequences or their complements to the reference genomic sequence. The nucleotides of the reference sequence aligning to these nucleotide sequences or their complements will be assigned to their respective RRD; the RRDs are assigned at (genomic) base-pair resolution. In case the sample is an environmental sample, the resulting processed data obtainable by the method provided herein may for example be used for analysing the quantitative presence of different microbes in said environmental sample. The one or more reference nucleotide sequences of (b) are preferably of a particular part of the genomic sequence, such as the part encoding ribosomal RNA (such as nucleotide sequences of 16S rRNA genes in case screening for the presence and quantity of different microbes in an environmental or biological sample), and the nucleotide-sequencing dataset may be a quantitative DNA sequencing dataset or an RNA-seq data set. Optionally, the reference sequences may be RNA sequences (optionally 16S rRNA genes), and the nucleotide-sequencing dataset may be an RNA-seq data set. The person skilled in the art understands that possible applications of this method are not limited to the detection of different microbes. For instance, instead of the quantitative presence of different microbes, the quantitative presence of two or more (different) cell types may be analysed. Different cell types may be cells from different individuals, e.g. in case of a sample of a pregnant animal or human the nucleic acid sample of the method provided herein may comprise cells of the mother and the child, foetus or embryo. In addition or alternatively, different cell types may be cells from different tissue, optionally from the same individual, e.g. from different organs, or from different health status (e.g. healthy cells versus cancerous and/or metastatic cells). As another non-limiting example, the quantitative presence of a pathogen may be determined, e.g. in a biological or an environmental sample. Optionally, (parts of) a parasite, bacterium or virus may be quantitatively detected in a biological or an environmental sample, such as, but not limited to, the quantitative detection of Covid-19 in a biological or an environmental sample.

The nucleotide-sequencing dataset of (a) may be a transcriptomic dataset comprising read depth values of RNA, reflecting the amount of transcripts or RNA species present in a nucleic acid sample. Transcriptomic data may be obtained by (massive parallel) RNA sequencing (RNA-seq) or any other method providing quantitative expression level data. The transcriptomic dataset may be obtained by RNA-seq. Preferably, RNA-seq is performed without an amplification step in order to avoid amplification bias. Such amplification free method may be based on single-molecule-based platforms such as PacBio single-molecule real-time (SMRT) sequencing (Kukurba and Montgomery, Cold Spring Harb Protoc. 2015 November; 2015 (11): 951-969). Optionally the transcriptomic dataset provides for the presence and quantity of total RNA-transcripts or a specific subset thereof such as (pre-) mRNA or any one of the other subsets such as, but not limited to, rRNA, tRNA, snRNA, snoRNA, miRNA, piRNA, tasiRNA, lncRNA and combinations thereof of said provided sample, preferably obtained by RNA-seq. Preferably, said provided sample has been processed to allow for RNA-sequencing, preferably to allow for sequencing of mRNA or any of the other RNA-transcripts. Preferably (part of) the genome of the provided sample may be used as the reference nucleotide sequence in step (b) of the method provided herein.

Preferably, the step (a)(1) of providing a nucleic acid sample comprises a step of sample collection and optionally nucleic acid, DNA and/or RNA extraction, enrichment and/or isolation of said sample, wherein said nucleic acid, DNA and/or RNA is subsequently subjected to nucleic acid, DNA and/or RNA sequencing thereby providing a quantitative nucleic acid, DNA and/or RNA (RNA-seq) dataset of said sample. The skilled person is aware of techniques suitable for extraction, enrichment and/or isolation of total nucleic acid molecules (DNA and/or RNA) and/or a specific type or subset of said nucleic acid molecules. In case of DNA, the extraction may be the extraction of cellular DNA or cell free DNA for instance from samples of bodily fluids such as blood or plasma, or environmental samples. The person skilled in the art is well aware of methods to extract DNA. Cellular DNA extraction may comprise a step of cell lysis using detergents and surfactants, protein and RNA degradation, ethanol precipitation, phenol-chloroform extraction and/or (mini) column purification. Many DNA extraction kits are available for specific samples including for e.g. plant cells, tissues and soil. Some RNA extraction methods, e.g. phenol extraction and extraction using commercially available kits (e.g., Qiagen RNeasy Kit; Zymo Research Direct-zol) have been described in Scholes and Lewis, BMC Genomics (2020) 21:249, which is incorporated herein by reference. The enrichment and/or isolation may be for a subset of nucleic acid molecules or DNA or RNA fractions, for instance in case of RNA the mRNA fraction. Enrichment and/or isolation of mRNA from a nucleic acid sample may be performed by selectively capturing poly-A tailed RNAs from said sample, for instance by using magnetic beads conjugated with poly(dT) oligonucleotide. In addition or alternatively, other DNA and/or RNA subsets may be of interest and can be enriched and/or isolated in step (a)(2) of the method provided herein. The skilled person is aware of suitable methods for enriching and/or isolating different RNA subsets, e.g. size fractionation by gel electrophoresis, silica spin columns for binding and elution of small RNA, or methods making use of difference in solubility properties of (over-dried for 1-24 hours) pelleted RNA in water between small (preferably <100 nucleotides are releasable) and large RNA molecules (no longer solubilized) including mRNA and rRNA (Choi et al. RNA Biol. 2018; 15(6): 763-772). In addition or alternatively, the method may comprise a step (a)(2) of enriching DNA and/or RNA subsets that comprise and/or anneal to a specific sequence, for instance by using techniques like capture probe hybridization, e.g., by using magnetic beads conjugated with capture probes or oligonucleotides comprising a sequence that is capable of annealing to a DNA and/or RNA subset.

Optionally, the nucleotide-sequencing dataset is an RNA-dataset, preferably a quantitative RNA-dataset comprising read depth values of one or more RNA sequences. Hence, preferably, the nucleotide sequence dataset may be a transcriptomic dataset, and the method provided herein may be phrased as a method of processing transcriptomic data of a nucleic acid sample, wherein the method comprises the steps of:
(a) obtaining a transcriptomic dataset comprising read depth values assigned to nucleotide sequences of transcripts present in the sample:
(b) obtaining a reference genomic nucleotide sequence;
(c) converting the read depth values present in the dataset to single characters indicating RRDs by ordinal class assignment; and
(d) mapping the single characters to the reference nucleotide sequence of (b) at base-pair resolution.

Preferably, sequences of reference genome are transcribed into transcripts of the transcriptomic dataset as defined herein. Optionally, in RNA-seq, the transcribed RNA (optionally enriched and/or isolated RNA) can be converted into complementary DNA (cDNA). The cDNA is subsequently processed to form a sequencing library for (deep-) sequencing. The person skilled in the art is aware how to handle and process samples for RNA sequencing. Said RNA sequencing is preferably mRNA (deep-) sequencing. Hence, in case of RNA, preferably, the RNA provided in step (a)(1) or (a)(2) is converted to cDNA and in step (a)(3) a sequencing library is prepared of said cDNA. Optionally, the cDNA is amplified prior to sequencing, optionally using primers comprising an UMI. The sequencing data obtained in step (4) of said cDNAs correspond to the originating RNAs. Optionally, step (b) of obtaining reference nucleotide sequence is performed by sequencing. Optionally, method provided herein comprises the step (a)(1) to (a)(3) indicated above, and the sample provided in step (a)(1) is split into at least two fractions, wherein one fraction of the sample is subjected to steps (a)(3)-(a)(5), and optionally step (a)(2) as defined herein, and another fraction of the sample is subjected to genomic sequencing. Said sequencing may be the sequencing of a target genomic sequence and/or a genomic sequence of interest. Preferably, said genomic sequencing is whole genome sequencing. The genomic sequence obtained may serve as a reference sequence in step (b) of the method provided herein. Optionally, prior to genomic sequencing, (part of) the sample is processed, wherein said processing preferably comprises subjecting the sample to DNA enrichment and/or isolation, and sequencing library preparation, wherein said sequencing library is subsequently subjected to (whole)genomic sequencing.

Hence, in an embodiment, the method provided herein comprises steps (a)(1) to (a)(3) as defined herein, wherein step (1) further comprises aliquoting the provided sample into at least two parts (or fractions), wherein said first part is subjected to genome analysis in order to obtain a reference sequence to be provided in (b) and said second part is subjected to transcriptome analysis in step (a)(3) and optionally step (a)(2), as defined herein in order to obtain a nucleotide-sequencing database to be provided in (a). Preferably, the method comprises a step (a)(2), wherein a part of the sample is enriched for the mRNA fraction. Optionally, the part of the sample that is subjected to genome analysis, for obtaining the reference nucleotide sequence in step (b), comprises a step of DNA enrichment and/or isolation prior to the step of genome sequencing.

Preferably, said DNA sequencing is (whole)genomic DNA sequencing. Hence, the sample of the method provided herein comprises nucleic acids, wherein said nucleic acids preferably comprises DNA and RNA, wherein said nucleic acids preferably comprises genomic DNA and mRNA. In an alternative embodiment, a reference (whole or partial) genomic sequence used in step (b) of the method provided herein may be obtained from a sequence library and/or public database. Said reference genomic sequence may be the genomic sequence as publicly available from the particular species from which the sample is derived. For instance, if the sample is from a human, a publicly available human reference genomic sequence may be used. As another non-limiting example, in case the sample is a Heinz tomato, a publicly available reference genomic sequence of Heinz tomato may be used.

The nucleotide-sequencing dataset of step (a) comprises read depth values. Preferable, a read depth per nucleic acid or per nucleotide (DNA or RNA) reflects the amount of said specific nucleic acid (DNA or RNA) present in the sample. In case of RNA-seq, the read depth value may reflect the transcript expression levels in the tissue and/or cell(s) from which the sample is derived. Read depths are arbitrary values that may range from 0 (i.e. not present in the nucleotide-sequencing dataset) to high multiple digit numbers as there is a very wide range of DNA and/or RNA present in the sample, for instance, in case or RNA seq, there is a wide range of RNA expression levels across the genome.

Conversion of read depth values into single characters according to the method provided herein allows for the capturing of read depth information at base-pair resolution, optionally across the entire genome, without requiring normalization. Using single characters for read depth values minimizes data storage space and computational requirements when using the data for further processing such as for deep learning, while retaining as much of the nucleotide sequencing data as possible. In case the nucleotide sequencing data is RNA-seq data, and the reference nucleotide sequence of is a genomic sequence, the RRDs obtainable by the method of the invention may represent relative expression of said genomic base pair in the sample. In case the sample is a sample from an individual, the method provided herein may result in the representation of the genomic sequence by a strand of characters (RRDs) bearing expression information at base-pair resolution. In case the nucleotide sequencing data is obtained by quantitative genomic DNA sequencing, and the reference nucleotide sequence is a genomic sequence, the RRDs may represent the quantitative presence of said genomic base pair in the sample. In case the sample is an environmental sample, the method provided herein may result in the representation of one or more genomic sequences by a strand of characters (RRDs) bearing information on the quantitative presence or absence of genomic DNA at base-pair resolution.

The resulting strand of single characters (RRDs) can be used in analysis, preferably in (computer-implemented) analysis of (massive) nucleic acid sequencing datasets, e.g. (massive) RNA-seq datasets and/or (massive) quantitative DNA sequencing datasets. The resulting strand of single characters can also be used in artificial intelligent processes or methods for instance for predicting genes, exons and/or introns within the genomic sequence. Hence, the method provided herein allows for transcriptomic data to be mapped easily to genomic sequences, which can straightforwardly be used for further processing such as deep learning, machine learning and/or artificial intelligence processes and/or applications.

Any system of single characters can be suitable for use in step (c) of the method of the invention.

Although not limited thereto, it can be straightforward to use the (Arabic) numeral single digits, i.e. the numbers selected from the group consisting of {0, 1, 2, 3, 4, 5, 6, 7, 8, 9}, to represent the RRDs as the ranking from low to high is inherent and intuitive, i.e. 0 being the lowest ranked single digit and 9 the highest ranked single digit. Optionally, the total number of categories represented by the RRDs in the class assignment model is ten. Although in principle arbitrary single characters can be used for each particular RRD, in case in total ten RRDs are used, (Arabic) numeral single digits may be used, and, although not limited to such system, the RRDs consecutively ranked from low to high may be assigned 0, 1, 2, 3, 4, 5, 6, 7, 8 and 9, wherein the lowest range is assigned number 0, the subsequent range is assigned number 1, up until the highest range that is assigned number 9.

Apart from the ten single character signs of the Arabic numerical system, there are many suitable alternatives. For instance, (part of) the 52 characters of the (Latin) alphabet, the 94 visible sings of the ASCII-table, Unicode characters may or the like be used. For instance, if the read depth values are categorized over in total 52 RRDs, for instance all 52 letters of the alphabet can be used to indicate the RRDs, wherein optionally the ranking of the order of letters in the alphabet may be adhered (i.e. ‘a’ for the lowest ranked RRD, ‘z’ for the highest ranked RRD). Likewise, other single character systems may be used, e.g. the signs of the ASCII-table, Unicode characters and the like. The skilled person understands that these numbers per system (e.g. Arabic numbers, Latin alphabet, signs of the ASCII-table, Unicode characters and the like) can be multiplied easily for instance by using different colours, different size of the signs, or, in case of alphabetic letters, using small caps, capital letters and/or different font types.

The number of RRDs or single characters is lower, preferably substantially lower, than the maximum read depth of the obtained nucleotide-sequencing data set. Preferably, the number of RRDs used in the method provided herein is at least about 5, 10, 100, 500, 1000, 5000, 10000, 50000 or at least about 100000 times lower than the maximum read depth of an obtained dataset.

It is understood by the person skilled in the art, that if it is desired to divide the read depth values of the nucleotide-sequencing data in less than the total number of characters of a specific system, for instance less than 52 ranges in case the Latin alphabet is used, part of the system may be used, for instance less letters of the alphabet may be used as single characters for the RRDs. Likewise, if it is desired to divide the read depth values of the nucleotide-sequencing data in a number exceeding the total number of characters of a certain system, combinations of systems may be used. For instance in case the read depth values would be divided over more than 52 RRDs, the alphabet may be used in combination with for instance the Arabic numerical system. It is understood by the skilled person that any other (combination or partial) system of distinguishable single characters can be used.

Assignment of read depth values to RRDs may be performed by first setting the boundaries for each RRD. Independent of the single character system for use in step (c) of the method of the invention, each RRD ranked from low to has a ranking value. For calculation of the boundaries herein, ranking values each are assigned a natural number (i.e. selected from the set {0, 1, 2, 3, 4 . . . N}), wherein N is the highest ranking value and the total number or RRDs is given by N+1 (i.e. in case of a total number of 10 RRDs, the RRD with the highest ranking value has ranking value 9.

Preferably, the read depth values of a nucleotide-sequencing dataset are converted to RRDs by class assignment, wherein the total number of RRDs is ten. Although the final single character used for each RRD is irrelevant as long as each RRD receives its own destined single character, the inventors developed an algorithm calculating the boundaries for each of the RRDs based on the ranking of these RRDs (indicated herein as the ranking value of the RRDs), wherein RRDs ranked from low to high receive the consecutive ranking values 0, 1, 2, 3, 4, 5, 6, 7, 8 and 9, respectively, i.e. 0 being the lowest ranked RRD, 9 being the highest ranked RRD.

Preferably, the read depth values of a nucleotide-sequencing dataset are converted to RRDs by class assignment, wherein the total number of RRDs is ten. Although the final single character used for each RRD is irrelevant as long as each RRD receives its own destined single character, the inventors developed an algorithm calculating the boundaries for each of the RRDs based on the ranking of these RRDs (indicated herein as the ranking value of the RRDs), wherein RRDs ranked from low to high receive the consecutive ranking values 0, 1, 2, 3, 4, 5, 6, 7, 8 and 9, respectively, i.e. 0 being the lowest ranked RRD, 9 being the highest ranked RRD. Since these ranking values themselves are single digits, optionally the single characters used to indicate the RRDs in step (c) of the method of processing nucleotide-sequencing data as provided herein are identical to the ranking values of these RRDs. In other words, the ranking value of an RRD may be identical to (the single character of) the RRD. However, the skilled person understands that these ten ranking values may be transformed in any other ten-fold single character system. As a non-limiting example, the RRD with ranking value 0 may be indicate with character ‘a’, the RRD with ranking value 1 may be indicated with character ‘b’, the RRD with ranking value 1 may be indicated with character ‘c’, etcetera.

As indicated above, each RRD has a certain read depth upper boundary, and read depth values exceeding said upper boundary will be assigned to a higher ranked RRD. In other words, read depth values will be assigned to the lowest ranked RRD for which the read depth value does not exceed the read depth upper boundary.

In principle, any model used to fit read count values over RRD can be used, such as, but not limited to a linear, exponential, exponential, polynomial, logarithmic, power, etc. Preferably, the model chosen depends on the nature of the data of the dataset, and a model is used in order to capture as much information as possible. In case the datasets are quantitative DNA sequencing datasets reflecting the amount and type of genomic DNA of one or more species present in for instance an environmental sample, a linear model may be used. In a linear model, the read count values are divided evenly over the total number of RRDs. In case the dataset is a RNA-seq dataset reflecting the amount and type of genes expressed from the genome, an exponential model may be used.

Preferably, independent of the model chosen for class assignment, a “zero dogma” is applied. This can be explained as follows. Some nucleotides of the reference nucleotide sequence may not be represented or present in the nucleotide-sequencing dataset. For instance in the sample from which the nucleotide-sequencing dataset is obtained which may be an RNA-seq dataset, some nucleotide sequences of the genome will not be expressed, or will be expressed a level below detection. Hence, the “zero dogma” is to be understood herein as the assignment of the nucleotides of the reference nucleotide sequence for which no data is present in the nucleotide-sequencing dataset, or for which zero transcripts are expressed, to the lowest ranked RRD. Although not limited thereto this lowest ranked RRD may be assigned the single character ‘0’. Preferably, nucleotides of the reference nucleotide sequence for which any read count value above zero is found in the nucleotide-sequencing dataset are assigned to the higher ranked RRDs based on an class assignment model.

In a linear regression model, read count values above zero to the maximum read count value of a dataset may be divided in an equal fashion over the remaining RRDs. Ranking values of RRDs may be used in order to define upper and/or lower boundaries of the RRDs. For instance, in ranking RRDs from low to high, the lowest ranked RRD may be assigned the ranking value 0, and any subsequently ranked RRD is assigned a ranking value that is 1 higher than the ranking value of the previous RRD. For instance, in case of 10 RRDs, the RRDs ranked from low to high may be assigned a ranking value of 0, 1, 2, 3, 4, 5, 6, 7, 8, and 9, respectively. Using such ranking value for RRDs, the following formula may be used for calculating the upper boundaries of each RRD according to a linear class assignment model:

$$UB = ( RV / N ) * MRD \qquad ( Formula 1 )$$

wherein:
UB is the read depth upper boundary of the given RRD;
RV is the given ranking value selected from the group consisting of {0, 1, 2, 3, 4, . . . N} of the RRD for which the upper boundary is calculated;
N is the ranking value of the highest ranked RRD; and
MRD is the maximum read depth.

In a non-limiting example, the upper boundaries in case N is 9, and the total number of RRDs is 10 (N+1), and the MRD=9000, read depth values will be assigned a certain RRD if they are within the domain of said RRD, which are indicated in Table 1.

TABLE 1
RRD ranking values and their read depth value domains according to
the linear class assignment model provided herein, wherein the highest
ranked RRD has a ranking value of 9, and the MRD = 9000.

| RRD ranking value | Read depth value domain |
|---|---|
| 0 | [0] |
| 1 | <0, 1000] |
| 2 | <1000, 2000] |
| 3 | <2000, 3000] |
| 4 | <3000, 4000] |
| 5 | <4000, 5000] |
| 6 | <5000, 6000] |
| 7 | <6000, 7000] |
| 8 | <7000, 8000] |
| 9 | <8000, 9000] |

In the Example of Table 1, the upper boundary of a certain RRD is to be understood as the maximum value that is still within the RRD. However, the skilled person will understand that also an upper boundary may be decided to be the lowest value of the subsequently higher ranked RRD. If the latter is the case, the domain of for instance RRD with ranking value 1 has to be indicated as [1000, 2000>. As will be obvious for the skilled person, what systematic is used is not relevant, as long as the same systematic is used throughout.

It is to be understood herein that the opposite way of ranking is also feasible and has the same end result: each RRD has a certain read depth lower boundary, and read depth values equal to or lower than said lower boundary will be assigned to a lower ranked RRD. In this way of ranking, read depth values will be assigned to the highest ranked RRD for which the read depth is not equal to or below the read depth lower boundary. For ranking, it may be sufficient to identify each of the RRD upper boundaries. Likewise, it may be sufficient to identify each of the RRD lower boundaries.

Optionally, an exponential class assignment model is used. Such exponential model is in particular suitable for processing RNA-seq data. Within such model, the lowest ranked RRD may have a read depth lower and upper boundary of 0 (zero dogma), the highest ranked RRD may have a read depth upper boundary of the maximum read depth of the nucleotide-sequence dataset and any one of the intermediate ranked RRDs may have a read depth upper boundary that is provided by the formula:

$$UB = f \cdot {x}^{RV} \qquad ( Formula 2 )$$

wherein:
UB is the read depth upper boundary of the given RRD;
x is a base that preferably has a value between 1 and Euler's number (e);
f is a scaling factor that preferably has a value of at least 1;
RV is the given ranking value selected from the group consisting of {1, 2, 3, 4, . . . N} of the RRD for which the upper boundary is calculated; and
N is the ranking value of the highest ranked RRD.

Euler's number (e) is known as being 2.718281 . . . According to this exponential model, the lowest ranked RRD has a lower and upper boundary of 0 (zero dogma), and any subsequently ranked RRD has a lower boundary that is equal to the upper boundary of the lower ranked RRD, and the total number of RRDs is N+1. Hence, preferably each of the RRDs ranked higher than the lowest ranked RRD has a read depth lower boundary that is provided by the formula:

$$LB = ( f \cdot {x}^{RV - 1} ) \qquad ( Formula 3 )$$

wherein:
LB is the read depth lower boundary of the given RRD;
x is a base that has a value selected from the range of 1 up and including Euler's number (e);
f is a scaling factor that has a value of at least 1;
RV is the given ranking value selected from the group consisting of {1, 2, 3, 4, . . . N} of the RRD for which the upper boundary is calculated; and
N is the ranking value of the highest ranked RRD.

Hence, in a preferred embodiment, read depth values will be assigned a certain RRD if they are within the domain of said RRD according to the exponential model of Formula 2 and 3, which are indicated in Table 2.

TABLE 2
RRD ranking values and their read depth value domains
according to the exponential model as provided herein,
wherein f, x, MRD and N are as defined herein.

| RRD ranking value | Read depth value domain |
|---|---|
| 0 | [0] |
| 1 | <0, f · x] |
| 2 | <f · x, f · x²] |
| 3 | <f · x², f · x³] |
| 4 | <f · x³, f · x⁴] |
| 5 | <f · x⁴, f · x⁵] |
| 6 | <f · x⁵, f · x⁶] |
| 7 | <f · x⁶, f · x⁷] |
| 8 | <f · x⁷, f · x⁸] |
| 9 | <f · x⁸, f · x⁹] |
| . . . | . . . |
| N | <f · x^{N-1}, MRD] |

Base x of the exponential model of Formula 2 and 3 may be provided by the formula:

$$x = {( MRD / f )}^{1 / N} \qquad ( Formula 4 )$$

wherein:
x is the base of Formula 2 and 3;
MRD is the maximum read depth;
f is the scaling factor of Formula 2 and 3; and
N is the ranking value of the highest ranked RRD.

Scaling factor f of the exponential model of Formula 2, 3 and 4 may have a value that depends on the maximum read depth (MRD) of the dataset. More in particular, f may be provided by the formula:

$$f = MRD / {e}^{N} \qquad ( Formula 5 )$$

wherein:
f is a scaling factor of Formula 2, 3 and 4;
MRD is the maximum read depth;
e is Euler's number; and
N is the ranking value of the highest ranked RRD.

In case base x and scaling factor f are defined according to Formula 4 and 5, the formula of calculating the UB of an RRD follows from integrating Formula 4 and 5 in Formula 2, which results in the following:

$$UB = ( MRD \cdot {e}^{RV} ) / {e}^{N} \qquad ( Formula 6 )$$

wherein
UB is the read depth upper boundary of the given RRD;
MRD is the maximum read depth
RV is the given ranking value selected from the group consisting of {1, 2, 3, 4, . . . N} of the RRD for which the upper boundary is calculated; and
N is the ranking value of the highest ranked RRD.

Likewise, in case base x and scaling factor f are defined according to Formula 4 and 5, the formula of calculating the LB of an RRD follows from integrating Formula 4 and 5 in Formula 3, which results in the following:

$$LB = ( MRD \cdot {e}^{RV - 1} ) / {e}^{N} \qquad ( Formula 7 )$$

wherein
UB is the read depth upper boundary of the given RRD;
MRD is the maximum read depth;
RV is the given ranking value selected from the group consisting of {1, 2, 3, 4, . . . N} of the RRD for which the upper boundary is calculated; and
N is the ranking value of the highest ranked RRD.

In an embodiment, f has the value of Formula 5, unless the maximum read depth (MRD) of a dataset is less than the value e^{N} (i.e. wherein MRD<e^{N}, wherein e is Euler's number and N is the ranking value of the highest ranked RRD), in which case f may have the value of 1. This may be the case for dataset from samples with low amount of starting material, such as, but not limited to single-cell RNA-seq datasets. For instance, within this embodiment, and in case N=9, f may have the value of 1 for a dataset in which the maximum read depth (MRD) that is lower than 8103, and f may have the value defined by Formula 5 in case a dataset has a maximum read depth of at least 8103. Hence, within this embodiment, f has the value of Formula 5 in case MRD≥e^{N} and the Upper Boundary can be calculated using Formula 6, and f preferably has the value of 1 in case MRD<e^{N}. Within this embodiment, and in case MRD<e^{N}, the formula of calculating the UB of an RRD follows from integrating Formula 4 and f=1 in Formula 2, which results in the following:

$$UB = {( MDR )}^{RV / N} \qquad ( Formula 8 )$$

wherein
UB is the read depth upper boundary of the given RRD;
MRD is the maximum read depth;
RV is the given ranking value selected from the group consisting of {1, 2, 3, 4, . . . N} of the RRD for which the upper boundary is calculated; and
N is the ranking value of the highest ranked RRD.

Likewise, within this embodiment, and in case MRD<e^{N}, the formula of calculating the LB of an RRD follows from integrating Formula 4 and f=1 in Formula 3, which results in the following:

$$LB = {( MDR )}^{RV - 1 / N} \qquad ( Formula 9 )$$

wherein
UB is the read depth upper boundary of the given RRD;
MRD is the maximum read depth;
RV is the given ranking value selected from the group consisting of {1, 2, 3, 4, . . . N} of the RRD for which the upper boundary is calculated; and
N is the ranking value of the highest ranked RRD.

Hence, in a preferred embodiment, the upper boundary of an RRD can be calculated by Formula 6 and the lower boundary of an RRD can be calculated by Formula 7 for a dataset that has a maximum read depth of at least e^{N} (for instance, in case N=9, MRD≥8103). In case the a dataset that has a maximum read depth of less than e^{N} (for instance, in case N=9, MRD<8103), the upper boundary of an RRD can be calculated by Formula 8 and the lower boundary of an RRD can be calculated by Formula 9.

Read depth values will be assigned a certain RRD if they are within the domain of said RRD. In a preferred embodiment, N=9, and the domains of the RRD are indicated in Table 3.

TABLE 3
RRD ranking values and their read depth value domains according
to the exponential model provided herein, wherein MRD is the
maximum read depth of the nucleic acid dataset, and wherein
the ranking value of the highest ranked RRD (N) is 9.

| RRD | Read depth value domains for | Read depth value domains for |
|---|---|---|
| ranking | datasets having a MRD of less | datasets having a MRD of at |
| value | than e⁹ | least e⁹ |
| 0 | [0] | [0] |
| 1 | <0, (MRD)^{1/9}] | <0, (MRD · e)/e⁹] |
| 2 | <(MRD)^{1/9}, (MRD)^{2/9}] | <(MRD · e)/e⁹, (MRD · e²)/e⁹] |
| 3 | <(MRD)^{2/9}, (MRD)^{3/9}] | <(MRD · e²)/e⁹, (MRD · e³)/e⁹] |
| 4 | <(MRD)^{3/9}, (MRD)^{4/9}] | <(MRD · e³)/e⁹, (MRD · e⁴)/e⁹] |
| 5 | <(MRD)^{4/9}, (MRD)^{5/9}] | <(MRD · e⁴)/e⁹, (MRD · e⁵)/e⁹] |
| 6 | <(MRD)^{5/9}, (MRD)^{6/9}] | <(MRD · e⁵)/e⁹, (MRD · e⁶)/e⁹] |
| 7 | <(MRD)^{6/9}, (MRD)^{7/9}] | <(MRD · e⁶)/e⁹, (MRD · e⁷)/e⁹] |
| 8 | <(MRD)^{7/9}, (MRD)^{8/9}] | <(MRD · e⁷)/e⁹, (MRD · e⁸)/e⁹] |
| 9 | <(MRD)^{8/9}, (MRD)] | <(MRD · e⁸)/e⁹, (MRD)] |

Table 4 provides preferred read depth value domains of the RRDs of the exponential model provided herein for N=9.

TABLE 4
RRD ranking values and their read depth value domains according to the
exponential model provided herein, for MRD = 500, 5000, 10000, 15000
or 20000, wherein the ranking value of the highest ranked RRD (N) is 9.

| RRD ranking | MRD = | MRD = | MRD = | MRD = | MRD = |
|---|---|---|---|---|---|
| value | 500 | 5000 | 10000 | 15000 | 20000 |
| 0 | [0] | [0] | [0] | [0] | [0] |
| 1 | <0, 2] | <0, 3] | <0, 3] | <0, 5] | <0, 7] |
| 2 | <2, 4] | <3, 7] | <3, 9] | <5, 14] | <7, 18] |
| 3 | <4, 8] | <7, 17] | <9, 25] | <14, 37] | <18, 50] |
| 4 | <8, 16] | <17, 44] | <25, 67] | <37, 101] | <50, 135] |
| 5 | <16, 32] | <44, 113] | <67, 183] | <101, 275] | <135, 366] |
| 6 | <32, 63] | <113, 292] | <183, 498] | <275, 747] | <366, 996] |
| 7 | <63, 126] | <292, 753] | <498, 1353] | <747, 2030] | <996, 2707] |
| 8 | <126, 251] | <753, 1941] | <353, 3679] | <2030, 5518] | <2707, 7358] |
| 9 | <251, 500] | <1941, 5000] | <3679, 10000] | <5518, 15000] | <7358, 20000] |

In addition to the single characters indicating the RRDs, further information may be added to the reference nucleotide sequence, preferably on base-pair resolution. For instance, information on modification, such as epigenetic modification, of the nucleotides may be added. This information may be captured in single characters as defined herein. Optionally, these characters are chosen from the same group of characters as used to identify the RRDs. Optionally, the ordering and/or specific location of this data may indicate whether it concerns a character representing an RRD or a character representing information on nucleotide modification. As an example, the RRD-indicating characters may be in a first row, and the nucleotide modification-indicating characters may be in a second row that is at a specific position in relation to the first row, for instance below the first row. As another example, for each nucleotide in the reference sequence, all characters indicating the different characteristics of each nucleotide (e.g. RRD and modification) are placed adjacent to one another on a fixed position. In such case, and in case of two different characteristics, the reference sequence is represented by a string of pairs of single characters, wherein the first character of a pair indicates one of the characteristics, and the second character of a pair indicates the other characteristic. Optionally, the characters indicating nucleotide modifications are chosen from a different group of characters as used for the RRDs. For instance, optionally, the RRDs are indicated by numbers (e.g. each RRD selected from the group 0, 1, 2, 3, 4, 5, 6, 7, 8 and 9) and the modification is indicated by a letter (for instance, ‘A’ for methylation, ‘B’ for glycosylation, ‘C’ for bond isomerization of uridine (forming pseudouridine) etc.). Optionally, further information may be added using further characters and/or (e.g. in case similar single characters are used) further positions. For instance, DNA-seq (such as CNV-seq) data may be added to RNA-seq data.

It is feasible to include further information to the same single character of the RRD. For instance, in case the total number of RRDs is 10, and there can be one nucleotide modification, all combinations can be indicated by a total number of 20 different single characters. As an example, in case a nucleotide is unmodified, depending on the RRD level, the nucleotide may initially be assigned an RRD selected from the group consisting of 0, 1, 2, 3, 4, 5, 6, 7, 8 and 9. In case a nucleotide is methylated, depending on the RRD level, the nucleotide may be assigned an RRD selected from the group consisting of a, b, c, d, e, f, g, h, i and j. The resulting string of RRDs may comprise a mixture of numbers and letters. The method therefore may further comprise a step of obtaining data on nucleotide modification. This data is preferably obtained or obtainable from the same sample as the sample providing the nucleotide-sequencing dataset comprising read depth values.

Further provided is the use of processed nucleotide-sequencing data obtainable by the method provided herein for further processing and/or analysis. For instance, in case of transcriptomic data, the processed data obtained by the method of the invention (preferably a string of RRDs mapped to a reference genomic sequence) is used for further processing and/or analysis such as comparing expression profiling between different samples, screening for alternative splicing, analysing allele-specific expression and/or predicting one or more genomic sequences. Preferably, the RRDs mapped to the reference genomic sequence at base-pair resolution as obtainable by the method of processing the transcriptomic dataset as obtained herein, are used for comparing expression patterns and/or profiling between multiple samples. The method provided herein may omit a further step of normalization, which is otherwise needed to correct for experimental variations, such as library fragment size, sequence composition bias, and read depth in order to accurately estimate gene and/or RNA transcript expression level values of different samples.

For further processing and/or analysis, multiple nucleotide-sequencing datasets may be combined. For instance, nucleotide-sequencing datasets of the same or similar individuals and/or derived from samples of the same or similar cells or tissues may be combined. Preferably, these multiple nucleotide-sequencing datasets are combined after processing by the method as provided herein. In other words, preferably the multiple nucleotide-sequencing datasets are each converted into RRDs and these RRDs are combined. Alternatively, the nucleotide-sequencing datasets are combined prior to processing by the method as provided herein. In other words, in such embodiment, the nucleotide-sequencing datasets are combined, preferably after normalization across the libraries based on the maximum reads per library, and subsequently converted into RRDs. Methods for normalizing across multiple nucleotide-sequencing datasets are known in the art, such as, but not limited to, Limma (Ritchie et al., Nucleic Acids Research, 2015, 43(7): e47), Combat (Johnson et al., Biostatistics, 2007, 8(1): 118-127; Leek et al., Bioinformatics, 2012, 28(6), pp. 882-883) and further methods described in Cole et al. (Cell Syst. 2019 April 24; 8(4): 315-328). Preferably, the processed nucleotide-sequencing datasets that are combined were mapped to the same reference nucleotide sequence in step (b) of the method provided herein. The combination of processed nucleotide sequencing datasets may result in complementation of RRDs for one or more nucleotide sequences of a reference nucleotide sequence e.g. for which data is only present in one of the combined datasets. Alternatively or in addition, such combination may also result in multiple RRDs for a certain reference nucleotide sequence base pair for which data is present in multiple of the combined datasets. Preferably, these multiple RRDs for a single reference nucleotide are reduced to a single RRD. In case the RRDs are present by numbers, reduction to a single value is preferred by calculating a mean or median that are rounded to a single digit character which is subsequently mapped to the reference nucleotide sequence. Preferably, there is an exception for the lowest ranked RRD. More in particular, if there is a combination of processed nucleotide-sequencing datasets and in one or more of these datasets a base pair of the reference sequence shows any reads (i.e. read depth higher than 0), said base pair will be assigned the penultimate lowest RRD in the reference sequence of the combined dataset.

In case the RRDs are presented by other characters, for instance the letters of the alphabet, these letters may be first converted to numbers corresponding to the ranking of each letter, a mean or median may be calculated that may subsequently be rounded, optionally to match a particular letter again, which letter is subsequently mapped to the reference nucleotide sequence.

Optionally, the nucleotide-sequencing data of the method provided herein is a part of a method for comparing nucleic acid molecule profiles between different samples. A profile is to be understood herein as information on nucleic acid type, nucleotide sequence, and/or quantity of nucleic acid molecules.

Optionally, the method of processing a nucleotide-sequencing dataset is part of a method to compare nucleic acid molecule profiles between different samples and/or the RRDs mapped to the reference nucleotide sequence at base-pair resolution as obtainable by the method of processing a nucleotide-sequencing dataset as defined herein is used for comparing nucleic acid molecule profiles between different samples. Optionally, the different samples are obtained from the same or a similar individual. The different samples may be obtained and/or collected from said individual at different time points, from different tissues and/or before and after (different) treatments. In an embodiment the method of processing a nucleotide-sequencing dataset is part of a method to compare nucleic acid molecule profiles of a same or similar individual under different circumstances. These different circumstances are almost endless, such as, but not limited to, a difference in exposure to biotic stress, abiotic stress, nutrients availability, water, toxins, daylight, etcetera, or to differences of the individual itself, such as age, disease, position of sample collection (for instance, in case of a plant, one sample may be a fruit sample and a further sample may be a root sample of the same plant). Alternatively, the different samples are obtained from different individuals, preferably from same or similar tissues, at the same or similar time point, and/or after the same or similar treatment.

Such method to compare nucleic acid molecule profiles between different samples preferably comprises the step of providing multiple different samples, and the steps (a), (c) and/or (d) may be performed on each sample in parallel or in serial. Optionally, step (a) is performed in parallel for multiple samples up to the sequencing step of step (a)(3) as defined herein, and optionally the nucleic acid molecules of each sample are labelled or tagged with a sample identifier sequence, and the samples and/or (isolated and/or enriched) tagged-nucleic acid molecules are subsequently pooled prior to subjecting the pooled samples to nucleotide sequencing. After sequencing, the data, preferably RNA-seq data, may be de-multiplexed based in the sample identifier sequence. The subsequent (de-multiplexed) RRDs are mapped to the reference nucleotide sequence, preferably genomic sequences relating to the original samples. In case of different samples from different individuals that are from the same species, the same reference nucleotide sequence, preferably reference genomic sequence, may be used for each such different individual.

Preferably, the RRDs mapped to the reference nucleotide sequence at base-pair resolution as obtainable by the method of processing the nucleotide-sequencing dataset as provided herein are used for comparing nucleic acid profiles between multiple samples. The method preferably omits a further step of normalization, which is otherwise needed to correct for experimental variability, such as sequence library fragment size, sequence composition bias, and read depth in order to accurately compare nucleic acid molecule levels of different samples.

In case of transcriptomic data, the method provided herein may be part of a method for comparing expression profiling between different samples.

Further, in case of a transcriptomic dataset, the method provided herein may be part of a method for predicting one or more genomic sequences. A gene sequence is to be understood as a sequence comprising an open reading frame. An open reading frame spans a sequence of genomic DNA between a start and stop codon that may be transcribed and subsequently processed to form mRNA. An open reading frame comprises one or more exons and optionally one or more introns. Prediction of a gene sequence may be the prediction of at least one exon, an intron, an exon and/or intron boundary, an open reading frame, a regulatory sequence and a whole gene sequence, wherein said whole gene sequence comprises an open reading frame and one or more transcription regulatory sequences, such as, but not limited to, a promoter sequence. Using RRDs mapped to a genomic sequence at base-pair resolution as obtainable by the method of processing a transcriptomic dataset as defined herein, unknown gene sequences can be predicted based on expression patterns as indicated by the RRDs mapped to known reference gene sequences for which the location in the genome is also known.

Optionally, provided herein is a process for predicting genes and gene expression levels of a sample, without obtaining a transcriptomic dataset for said sample. Preferably, said prediction is performed by machine learning. Therefore, also provided is a method of predicting one or more genomic sequences and optionally the gene expression levels of said genomic sequences, comprising the steps of:
(A) providing RRDs mapped to one or more reference genomic sequences at base-pair resolution as obtainable by a method of processing a transcriptomic dataset as provided herein;
(B) providing gene annotation data of at least part of said one or more reference genomic sequences;
(C) assigning by a training engine the gene annotation data of (B) to the RRDs mapped to the one or more reference genomic sequences of (A);
(D) training a machine-learning model, using the gene annotation data assigned in step (C) to the mapped RRDs, wherein during the training the machine-learning model learns to assign gene annotations to one or more further genomic sequences to which RRDs have been mapped; and
(E) obtaining the machine-learned model.

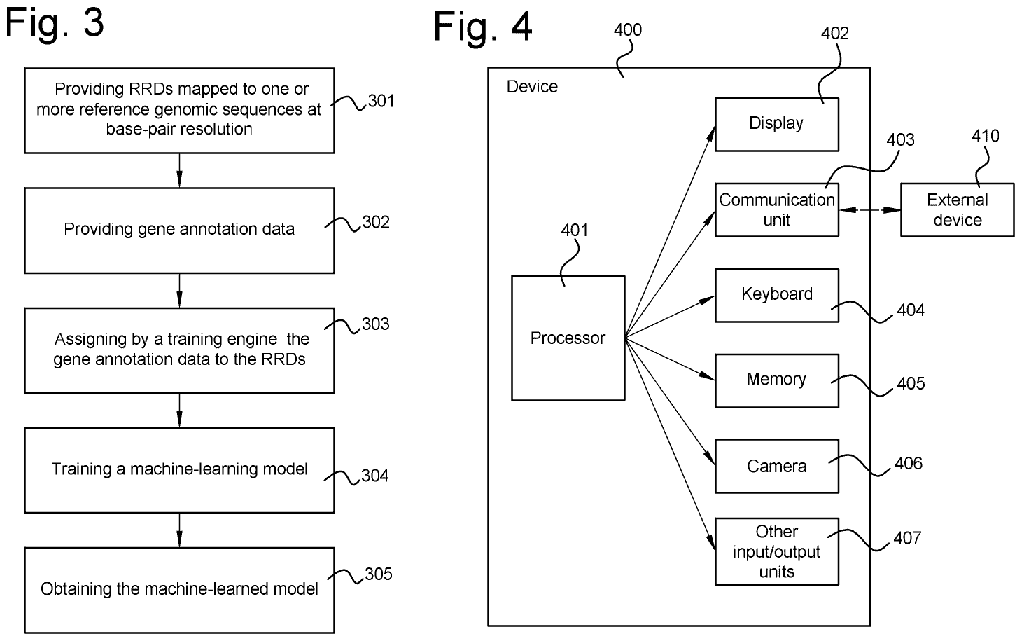

FIG. 3 depicts a flowchart illustrating said method, having a step 301 of providing RRDs mapped to one or more reference genomic sequences at base-pair resolution, a step 302 of providing gene annotation data of at least part of said one or more reference genomic sequences, a step 303 of assigning by a training engine the gene annotation data to the RRDs, a step 304 of training a machine-learning model, using the gene annotation data that was in step 303 assigned to the mapped RRDs, and a step 305 of obtaining the machine-learned model. Hence in FIG. 3, step 301 corresponds to step (A) as described herein, step 302 corresponds to step (B) as described herein, step 303 corresponds to step (C) as described herein, step 304 corresponds to step (D) as described herein and step 305 corresponds to step (E) as described herein.

The assigning by training engine in step (C) preferably is automated and/or performed automatically. Preferably, said method is a computer-implemented method. Optionally, the one or more further genomic sequences of step (D) are sequences related to the sequences for which gene annotation data is provided in (B) “Related” as used herein may mean that the genomic sequences are from samples that are of the same individual, from a different individual of the same species, or from an individual of a different species of the same genus, family or order. In addition, “related” as used herein may mean that the transcriptomic data mapped to the genomic sequence is obtained from an individual that has been treated similarly.

Optionally, the method provided herein is performed using multiple transcriptomic datasets from multiple samples and/or optionally multiple strings of RRDs mapped to a genomic sequence are obtained. Optionally, said multiple samples are highly comparable, preferably being from the same species, the same individual, the same tissue type, the same tissue, and/or the same cell type. Optionally, said multiple samples are from the same species, the same tissue type, but from different individuals. Optionally, said multiple samples are from the same individual but from different tissue types. Optionally, said multiple samples are from the same individual but from different cell types. Optionally, said multiple samples are from the same species and from same tissue, but the different individuals have been treated differently, for instance being mutagenized or not, being exposed to biotic stresses or not, being exposed to non-biotic stresses, etc. Optionally, said multiple samples are from the same species and from same tissue, but the different individuals are in a different stage of development, e.g. younger or older.

Optionally, the method of predicting gene sequences as defined herein is preceded by the method of processing transcriptomic data as defined herein. Optionally, the method of predicting gene sequences comprises predicting gene sequences and/or comparing gene expression levels of the multiple samples.

Optionally, the method of processing a nucleotide-sequencing dataset, the method of comparing nucleic acid molecule profiles and/or gene expression levels and/or the method of predicting genomic sequences is a computer implemented method. Preferably said computer is programmed to perform any one of the methods provided herein. Preferably the computer comprises a computer-readable storage means that comprises the (compressed) nucleotide-sequencing dataset processed according to the method provided herein. Also provided is a computer-readable means that is configured to perform a method of the processing a nucleotide-sequencing dataset, the method of predicting genomic sequences and/or the method of comparing nucleic acid molecule profiling and/or gene expression levels as provided herein. Also provided is a computer program product comprising instructions that, when executed by a processor system, cause the processor system to perform the method of the processing nucleotide-sequencing data, the method of predicting genomic sequences and/or the method of comparing nucleic acid molecule profiling and/or gene expression levels as provided herein. Preferably, the method as provided herein is a method performed by an electronic device.

Further provided is a storage means storing instructions, wherein the instructions are configured to cause a processor to perform the method of the processing nucleotide-sequencing data, the method of predicting genomic sequences and/or the method of comparing nucleic acid molecule profiling and/or gene expression levels as provided herein.

Preferably, the processed nucleotide-sequencing data obtained or obtainable by the method provided herein is stored on a computer-readable storage means. Also provided is a computer-readable storage means comprising the processed nucleotide-sequencing data obtained or obtainable by the method provided herein.

Further provided is the use of processed nucleotide-sequencing dataset obtained or obtainable by the method provided herein and/or the use of a computer-readable storage means comprising said processed nucleotide-sequencing dataset obtained or obtainable by the method provided herein for further processing and/or analysis, such as for predicting one or more genomic sequences and/or for comparing nucleic acid molecule profiling and/or gene expression profiling between different samples.

FIG. 4 shows a computing device 400, e.g., a mobile phone, tablet, laptop, desktop, a TV, or any other computer device, etc., to perform the present invention, for example, the methods in any of FIGS. 1, 2 and 3.

The device 400 may comprise at least one of a processor 401, a display 402, a communication unit 403, a keyboard, a memory 405, a camera 406 and other input/output units 407.

The processor 401 is configured to perform the program/instructions stored in the memory 405, e.g., via controlling other components such as the display 402, the communication unit 403, the keyboard 404, the memory 405, the camera 406 and other input/output units 407.

The display 402 may be controlled by the processor 401 to perform the all the displaying function (and/or input function if it is a touch screen) in the present invention, for example with the method in FIG. 1, displaying any of the nucleotide-sequencing dataset, reference nucleotide sequences, single characters indicating RRDs, and the mapping results; for example with the method in FIG. 2, displaying any of the subset, the sequencing library, and the read depth values; for example with the method in FIG. 3, displaying any of the RRDs, the one or more reference genomic sequences, and the gene annotation data. When the display 402 is a touch screen, it may be used to receive input of all the relevant information to perform the methods, for example the informed obtained in any of the “providing” steps in any of FIGS. 1 and 1 or any steps that requires a user input (e.g., in step 303 and/or 304), wherein the keyboard 404 may perform the same function.

The communication unit 403 may be controlled by the processor 401 to perform all communication function in the present invention. For example, if an external device 410 (e.g., an sever) is used to perform some functions in the steps of FIGS. 1 to 3 (e.g., step 101 may be performed on the local device 400 and the final step 104 in FIG. 1, but other steps in FIG. 1 may be performed on the external device 410, e.g., a server; or all the steps may be performed on the local device 400; or a part of the steps is performed on the local device 400 and the remaining part of the steps is performed on at least one or more external devices 410), messages may be communicated via the communication unit 403. Optionally, the datasets and other information used in the present invention may be stored in the external device 410 or in the memory of the local device 400, e.g., any of the nucleotide-sequencing dataset, reference nucleotide sequences, single characters indicating RRDs, the mapping result, the subset, the sequencing library, the read depth values, the RRDs, the one or more reference genomic sequences, the gene annotation data, and the training engine/model. If they are saved in the external device 410, they are communicated to the local device 400 via the communication unit 403.

The memory 405 may be configured to store the instructions to perform the methods of the present invention, for example, the information mentioned above.

The camera 406 may be configured to capture images or used as an input device to scan documents/images to obtain the above-mentioned information, which is optional in the present invention.

The other input/output units 407 may be configured to perform other or the same input/output functions of the present invention, for example, to receive user input for the assigning the training engine and training of the model.

The device 400 may be configured to use the processed nucleotide-sequencing data obtained or obtainable by any one of the methods in the present invention, in a computer implemented method, for example, for predicting genomic sequences and/or for comparing gene expression patterns within or between samples.

1. A method of processing a nucleotide-sequencing dataset, wherein the method comprises the steps of:
(a) providing the nucleotide-sequencing dataset comprising read depth values assigned to nucleotide sequences;
(b) providing a reference nucleotide sequence;
(c) converting the read depth values present in the nucleotide-sequencing dataset to single characters indicating Relative Read Depths (RRDs) by ordinal class assignment; and
(d) mapping the single characters to the reference nucleotide sequence of (b) at base-pair resolution,
wherein the method is a computer-implemented method.

2. The method of claim 1, wherein the nucleotide-sequencing dataset of (a) is a transcriptomic dataset obtained by RNA-seq.

3. The method of claim 1, wherein the nucleotide-sequencing dataset of (a) is a genomic dataset obtained by DNA sequencing.

4. The method of claim 1, wherein the reference nucleotide sequence is a genomic sequence, preferably a whole genomic sequence.

5. The method of claim 1, wherein
step (a) comprises the following (sub-)steps:
(1) providing a nucleic acid sample;
(2) optionally enriching and/or isolating a subset of nucleic acid molecules from at least part of said nucleic acid sample;
(3) preparing a sequencing library of the nucleic acid molecules of (or the enriched and/or isolated nucleic acid molecules of;
(4) obtaining nucleotide sequences by sequencing the sequencing library of; and
(5) assigning read depth values to the nucleotide sequences obtained in, thereby obtaining a nucleotide-sequencing dataset comprising read depth values assigned to the nucleotide sequences of the sequenced nucleic acid molecules.

6. The method of claim 5, wherein the nucleic acid molecules of and optionally the enriched and/or isolated nucleic acid molecules of are mRNA molecules, and wherein the method further comprises a (sub-)step of converting the mRNA to cDNA prior to step of preparing a sequencing library of said cDNA.

7. The method of claim 1, wherein the reference nucleotide sequence of (b) is obtained by (whole)genomic sequencing.

8. The method of claim 1, wherein in step (c) the read depth values are classified in N+1 consecutive RRDs, wherein each RRD comprises a read depth upper boundary, wherein a read depth value is assigned to the lowest ranked RRD for which said read depth value does not exceed the read depth upper boundary of said RRD, wherein the read depth upper boundary of the lowest ranked RRD is 0, and wherein for the remaining RRDs the read depth upper boundary is given by:
$$UB = f \cdot xRV \qquad ( Formula 2 )$$
wherein,
UB is the read depth upper boundary of the given RRD;
x is a base that preferably has a value between 1 and Euler's number (e);
f is a scaling factor that preferably has a value of at least 1;
RV is the given ranking value selected from the group consisting of {1, 2, 3, 4, . . . N} of the RRD for which the upper boundary is calculated; and
N is the ranking value of the highest ranked RRD.

9. The method according to claim 8, wherein the value of the base x is:
$$x = ( MRD / f ) 1 / N \qquad ( Formula 4 )$$
wherein:
x is the base of Formula 2 and 3;
MRD is the maximum read depth; and
N is the ranking value of the highest ranked RRD.

10. The method according to claim 8, wherein the value of the scaling factor f is given by:
$$f = MRD / eN \qquad ( Formula 5 )$$
wherein:
f is a scaling factor of Formula 2 and 4;
MRD is the maximum read depth;
e is Euler's number; and
N is the ranking value of the highest ranked RRD.

11. The method according to claim 8, wherein f is provided by Formula 5 in case MRD≥e^{N}, and f=1 in case MRD<e^{N}, and wherein the read depth upper boundary of the lowest ranked RRD is 0, and wherein for the remaining RRDs the read depth upper boundary is given by:
$$UB = ( MRD \cdot eRV ) / eN \qquad ( Formula 6 )$$
in case MRD≥e^{N}; and by
$$UB = ( MRD ) R V / N \qquad ( Formula 8 )$$
in case MRD<e^{N},
wherein
UB is the read depth upper boundary of the given RRD;
MRD is the maximum read depth;
e is Euler's number;
RV is the given ranking value selected from the group consisting of {1, 2, 3, 4, . . . N} of the RRD for which the upper boundary is calculated; and
N is the ranking value of the highest ranked RRD.

12. The method according to claim 1, wherein said method is performed on multiple samples.

13.-14. (canceled)

15. A computer-readable storage medium comprising the processed nucleotide-sequencing data obtained by the method of claim 1.

16. A computing device comprising at least one processor, wherein the processor is configured to perform any of the method in of claim 1.
